(19) United States
(12) Patent Application Publication
(10) Pub. No.: US 2013/0030460 A1
(43) Pub. Date: Jan. 31, 2013

(54) INTRAVASCULAR THROMBOEMBOLECTOMY DEVICE AND METHOD USING THE SAME

(71) Applicants: Michael P. Marks, Hillsborough, CA (US); Like Que, Livermore, CA (US)

(72) Inventors: Michael P. Marks, Hillsborough, CA (US); Like Que, Livermore, CA (US)

(21) Appl. No.: 13/191,306

(22) Filed: Jul. 26, 2011

(51) Int. Cl. A61F 2/01

(52) U.S. Cl. 606/200

(57) ABSTRACT

A device and a method for increasing or restoring a flow in a body lumen are provided. The device and the method may treat conditions related to a stroke, such as an ischemic stroke, by removing an occlusion from a blood vessel and/or reopen a blood vessel. The device may comprise a pusher tube and an expandable compartment. The expandable compartment may comprise a control element, a reconfigurable element, and supportive element. The supportive element is configured to adjust a radial force and a configuration of the reconfigurable element, thereby allowing highly efficient removal of an occlusion from a blood vessel and/or reopen a blood vessel with least or no damage to the body lumen.

### BACKGROUND OF THE INVENTION

1. Field of the Invention

The present disclosures are generally related to a device used in a blood vessel and a method of using the same.

2. Description of the Related Art

A variety of disease conditions can be caused, at least in part, by blockage or occlusions of blood vessels. A well-known example of such conditions includes, but is not limited to stroke. Other such conditions include a myocardial infarction, limb ischemia, occlusions of vascular grafts and bypasses, and venous thromboses.

A stroke is often referred as a “brain attack.” It often results in rapid and significant loss of brain function due to disturbance in the blood supply to the brain. As a result, inabilities in movement, use of language, vision and many other biological functions may be temporarily or irreversibly impaired. Strokes are either hemorrhagic (due to bleeding) or ischemic (due to inadequate blood supply). The majority of strokes are ischemic. It is estimated that about 700,000 ischemic strokes occur in the United States annually. The major causes of an ischemic stroke include thrombosis (clotting) in a blood vessel supplying the brain or an embolus from another source such as the heart going to a blood vessel supplying the brain. Sometimes a thrombosis occurs where there is a pre-existing stenosis of blood vessels in the brain, usually form atherosclerotic disease.

Treatments for acute ischemic stroke are concentrated on re-establishing blood flow to the brain as quickly as possible. They include the use of a drug such as tissue plasminogen activator (tPA), a thrombolytic agent (clot-busting drug). More recently devices such as the Merci thrombectomy device (Concentric Medical, Mountain View, Calif.) and the Penumbra suction thrombectomy catheter (Penumbra, Inc., Alameda, Calif.) have been approved by the Food and Drug Administration for thrombectomy in acute stroke. These devices do not always achieve complete recanalization. Sometimes they fail to open the vessel at all or may only partially open the vessel. They also may take some time to work, with multiple passes of the devices into the intracranial circulation needed before the vessel is reopened. There is a need for devices with high rates of complete recanalization performed in a more rapid manner.

### SUMMARY OF THE INVENTION

According to one aspect of the invention, a device for use in a body lumen is provided. The device may comprise a pusher tube and an expandable compartment. According to some embodiments, the expandable compartment may comprise a control element, which may comprise a proximal end and a distal end, a reconfigurable element, which may be associated with a supportive element and the control element, and a supportive element, which may be associated with the control element and the reconfigurable element. According to some other embodiments, the supportive element may be configured to adjust a radial force and a configuration of the reconfigurable element.

In the foregoing device, the control element may comprise a wire, cable, or braid in at least some embodiments. In some other embodiments, the reconfigurable element and/or the supportive element may comprise a plurality of wires. In still some other embodiments, the reconfigurable element can self expand into a relaxed expandable state to form a compartment or basket. In still some other embodiments, the reconfigurable element may comprise a plurality of cells, and a size of a cell and a thickness of wires surrounding the cell may vary within the reconfigurable element.

In some embodiments, the supportive element of the foregoing device may be in a form of strut that may be manufactured from a same piece of material as the reconfigurable element and automatically connected to the reconfigurable element. In some other embodiments, the supportive element may be in a form of wire mesh or a braid. In some alternative embodiments, the pusher tube may be connected to the expandable compartment. In still some other embodiments, the control element may be surrounded by the pusher tube and can move freely inside the pusher tube.

In some other embodiments, the supportive element of the foregoing device may comprise a first configuration and a second configuration, said first configuration having a smaller angle between the supporting element and the control element than the angle in the second configuration. In some of certain embodiments, the supportive element may comprise a first configuration and a second configuration, said first configuration having an outer diameter which is smaller than an outer diameter of the second configuration.

According to still some other embodiments, projection of the control element to a distal direction may cause transition of the supportive element to the first configuration such that the radial force of the reconfigurable element may be reduced. In some alternative embodiments, projection of the control element to a proximal direction may cause transition of the supportive element to the second configuration such that the radial force of the reconfigurable element may be increased. In certain aspects, the device may be configured to remove an occlusion blocking a blood vessel, to open a blocked section of a blood vessel and/or to increase a flow in a blood vessel.

According to certain aspects, a distal end of the reconfigurable element may be joined forming a closed-end reconfigurable element, or not jointed forming an open-end reconfigurable element. In some embodiments, the sides of the reconfigurable element may be joined forming a closed-sided reconfigurable element, or not joined forming an open-sided reconfigurable element. In some of certain embodiments, the supportive element may comprise a plurality of wires, and said wires may be extendible between the proximal and distal ends, and substantially surrounded by the reconfigurable element.

According to some other aspects, the reconfigurable element and the supportive element of the foregoing device may be in a form of wire mesh, which can be extendible between the proximal and distal ends, and the supportive element may be substantially surrounded by the reconfigurable element, thereby forming a double-layered reconfigurable element. In some embodiments, the reconfigurable element may comprise a plurality of linear wires aligned substantially in parallel and a plurality of wires in a substantially circular form, and the supportive element may comprise at least two wires that is associated with the reconfigurable element and the control element, thereby forming an umbrella-shaped expandable compartment.

According to still some other aspects, a distal end of the reconfigurable element and a distal end of the supportive element may not be connected and move independently. In some of certain embodiments, an atraumatic flexible coil may be attached to the distal tip of the reconfigurable element.

According to still some other aspects, the device may comprise a distal expandable structure and a proximal expandable structure. In some embodiments, the first expandable structure may comprise a reconfigurable element, a control element, and optionally a supportive element. In some other embodiments, the second expandable structure may comprise a reconfigurable element and optionally an enclosing element. In certain some embodiments, a distance between the distal and proximal expandable structures may be adjustable, i.e. one structure can be slide freely on a sliding component to change its distance from the other structure.

According to still some other aspects, a method of removing an occlusion present in a first position of a blood vessel is provided. The method may comprise introducing the foregoing device according to at least some embodiments into the blood vessel, locating the device at the first position of the blood vessel (e.g. the site of occlusion), adjusting the radial force and/or configuration of the reconfigurable element of the device, and removing the occlusion from the first location. In some embodiments, removing the occlusion may further comprise one or more selected from the group consisting of engaging the occlusion at least partially with the device, disassembling the occlusion into small-sized debris and collecting at least part of the debris, and expanding the area of the blood vessel. In some other embodiments, the method may be configured to be applied for treatment of stroke.

According to still some other aspects, a method of increasing a flow in a blood vessel is provided. The method may comprise introducing the foregoing device according to at least some embodiments into the blood vessel, locating the device at about the first position of the blood vessel that is in need of increasing the flow, and adjusting the radial force and/or configuration of the reconfigurable element of the device so as to expand an area of the first position.

According to still some other aspects, a method of removing an occlusion present in a first position of a blood vessel is provided. The method may comprise introducing the foregoing device according to at least some embodiments into the blood vessel, locating the device at about the first position of the blood vessel, supporting the expanded status of the reconfigurable element by extending a support element associated with the reconfigurable element by proximal movement of the control element, further supporting a more expanded status of the reconfigurable element by extending a support element associated with the reconfigurable element by proximal movement of the control element, grabbing the occlusion with the reconfigurable element in its expanded status, shifting the configuration of the reconfigurable element toward a more relaxed status by distally moving the control element, and removing the occlusion from the first location.

According to still some other aspects, a method of removing an occlusion present in a first position of a blood vessel is provided. The method may comprise introducing the foregoing device according to at least some embodiments into the blood vessel, locating the device distal to the position of the blood vessel, pulling the pusher tube proximally to grab the occlusion between the two expandable structures in its expanded status, supporting the expanded status of the reconfigurable element by extending a support element associated with the reconfigurable element by proximal movement of the control element, grabbing the clot with the proximal expandable structure, catching the clot debris with the distal expendable structure, optionally retrieving the device while pulling the control element to increase the radial force of the expendable structure and further support the more expanded status of the expandable structure, and removing the occlusion from the first location.

### BRIEF DESCRIPTION OF THE DRAWINGS

FIG. 1 shows a non-limiting illustrative example of a device according to some embodiments of the invention.

FIG. 2 shows another non-limiting illustrative example of a device according to some embodiments of the invention, particularly when it is located in a body lumen, and illustrates some non-limiting examples of a mechanism to remove an occlusion from and/or expands a blood vessel according to some embodiments of the invention.

FIG. 3 shows still another non-limiting illustrative example of a device according to some embodiments of the invention.

FIG. 4 shows still another non-limiting illustrative example of a device according to some embodiments of the invention.

FIG. 5 shows still another non-limiting illustrative device according to some embodiments of the invention.

FIG. 6 shows still another non-limiting illustrative example of a device according to some embodiments of the invention.

FIG. 7 shows still another non-limiting illustrative example of a device according to some embodiments of the invention.

FIG. 8 shows still another non-limiting illustrative example of a device according to some embodiments of the invention.

FIG. 9 shows still another non-limiting illustrative example of a device according to some embodiments of the invention.

FIG. 10 shows still another non-limiting illustrative example of a device according to some embodiments of the invention.

FIG. 11 shows still another non-limiting illustrative example of a device according to some embodiments of the invention.

FIG. 12 shows still another non-limiting illustrative example of a device according to some embodiments of the invention.

FIG. 13 shows still another non-limiting illustrative example of a device according to some embodiments of the invention.

FIG. 14 shows a non-limiting illustrative example of a process of making a device according to some embodiments of the invention.

FIG. 15 illustrate non-illustrative examples of an apparatus comprising a device according to some embodiments of the invention.

FIG. 16 shows still another non-limiting illustrative example of a device according to some embodiments of the invention.

FIG. 17 shows still another non-limiting illustrative example of a device according to some embodiments of the invention.

FIG. 18 illustrate non-illustrative examples of an apparatus comprising a device according to some embodiments of the invention.

FIG. 19 illustrates some non-limiting illustrative examples of a way that blood clot is removed or the vessel is expanded according to some embodiments of the invention.

### REFERENCE NUMERALS FOR DESIGNATING MAIN COMPONENTS IN THE DRAWINGS

5: Guide wire
10: Control element
20: Pusher tube
25: Introducer sheath
30: Microcatheter
35: Microcatheter hub
40: Expandable compartment
50: Luminal surface
60: Occlusion/Clot
410: Reconfigurable element
420: Supportive element
425: Enclosing element
430: Connector
431: Outer connector
432: Inner connector
440: Markers
450: Distal end connector
451: Supporting element distal connector
452: Supporting element outer distal connector
453: Supporting element inner distal connector
455: Connecter of proximal expendable structure
460: Proximal end connector
461: Outer proximal end connector
462: Inner proximal end connector
463: Connector joining media (adhesive, solder etc.)
470: Adjustment tube
471: sliding tube
475: long inner tube
480: Plateau position
490: Control element handle tubing
510: Connecting tubing
520: Connecting wire/stretch resistance wire
540: Coil
550: Distal expandable structure/Distal structure
560: Proximal expandable structure/Proximal structure
570: Joining media
580: Pusher tubing connecting points
495: Distal flexible coil

### DETAILED DESCRIPTION OF THE EMBODIMENTS

The present disclosure is generally related to a device used in a body lumen, such as a blood vessel, and a method of using the same. In some embodiments, the device may be positioned in the body lumen to dilate the lumen and/or remove an occlusion from the lumen. While the device is in the portion of the body lumen that is in need of treatment, an operator can maneuver the device to expand the lumen and/or engage the occlusion.

Some aspects of the present invention provide a device and a method that are configured to treat conditions in blood vessels which include, but are not limited to, a stroke. In some embodiments, the device and the method are configured to treat conditions related to an ischemic stroke by removing an occlusion from a blood vessel and/or reopen a blood vessel with some underlying stenosis to resume blood flow therein.

Non-limiting examples of blood vessels may include, an artery, a vein and surgically implanted grafts and bypasses serving as components of the circulatory system.

The term “occlusion” generally includes any matter partially or completely obstructing a lumen of the blood vessel. The occlusion slows or obstructs flow running through the lumen. Examples of the occlusion may include blood clots and atherosclerotic plaques present in the vessel as well as fat or foreign bodies.

The term “stroke” generally includes a condition(s) that is in part caused due to disturbance in blood supply to a brain. The disturbance can be caused by blockage (e.g. ischemic stroke) and/or hemorrhage (e.g. hemorrhagic stroke) of blood. In particular, an ischemic stroke can be caused due to partial or substantial occlusion of blood vessel. Treatment of the ischemic conditions can be applied to blood vessels present in the brain as well as in other tissues such as the heart. Accordingly, the device and method disclosed in this application are not limited to use in any particular organs but can be applied to any blood vessel of the body that needs dilation of the lumen or removal of occlusion to restore blood flow. In addition, the device and method according to the present invention can be used to treat venous occlusions which may result in other conditions besides ischemia.

Furthermore, many different modifications and alternations, which should be obvious to a person with ordinary skill in the art, can also be done without affecting the scope of the invention to properly serve the specific treatment conditions. Therefore, not only the examples disclosed in this application but also such an obvious modification and alteration should also be included in the scope of the invention.

One aspect of the present invention is related to a device for use in a blood vessel comprising a reconfigurable element, supportive element, a control element, a pusher tube etc. The control element, reconfigurable element, and supportive element form an expandable compartment.

The device can be introduced into the blood vessel through a catheter. The “catheter” generally includes a tubular structure that can be inserted into a body lumen, thereby allowing administration of a device and/or chemicals to a body area that needs treatment. The term “microcatheter” may refer to a catheter that is configured to be administered in a relatively small body lumen such as blood vessels.

The sizes of blood vessels vary enormously, from a diameter of about 0.03 inch (about 1 mm) in smaller arteries and veins to 1.0 inch (about 25 mm) in the aorta. Accordingly, in some embodiments, the diameter of the device may range from approximately 0.01 inch (about 0.25 mm) in collapsed state to 1.0 inch (about 25 mm) in expanded state.

In some embodiments, the control element may comprise a wire, braid, or cable and be configured to control a configuration of the expandable compartment. Various materials can be used to manufacture the control element, which may include metal and non-metal materials. Some non-limiting examples of metal materials for the control element may comprise nickel, titanium, stainless steel, cobalt, chrome and any alloys of the foregoing such as Nitinol (NiTi), or Cobalt Chromium alloys. In addition, any polymers or plastics which have desired properties of being the control element can be used for production of the same. Polymers include, but not limited to, Polyimide, PEEK (Polyether ether ketone), Nylon, PTFE (polytetrafluoroethylene), PET (Polyethylene terephthalate), Polypropylene, etc. Polymer coated metal including but not limited to, PTFE coated Stainless Steel, or PTFE coated NiTi can also be used as control element; The control element can also be made of composite materials, such as PTFE or FEP (Fluorinated ethylene propylene) tubing over NiTi wire, or PTFE or FEP tubing over Stainless Steel etc.

The diameter of the control element may range approximately from 0.001 inch to 0.10 inch.

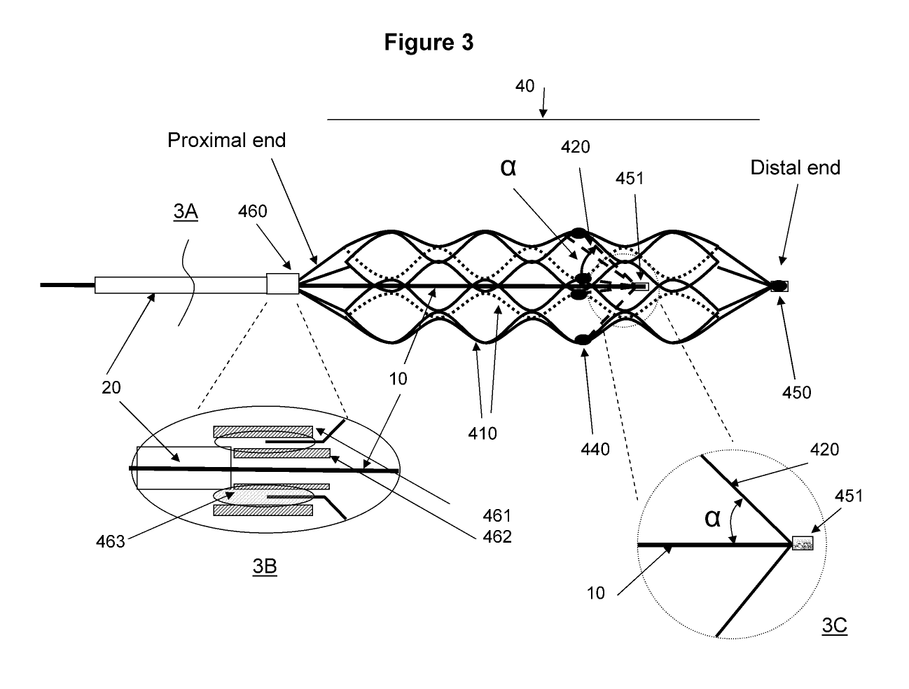

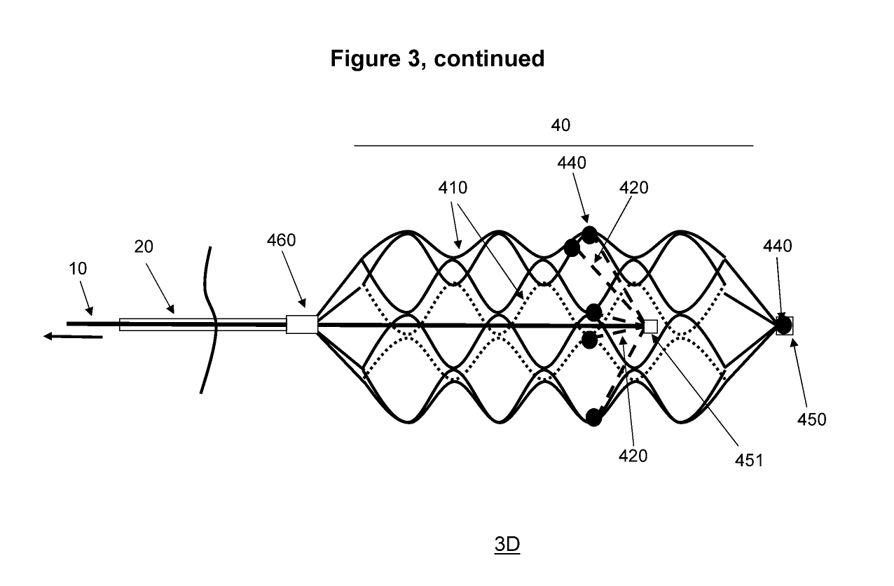

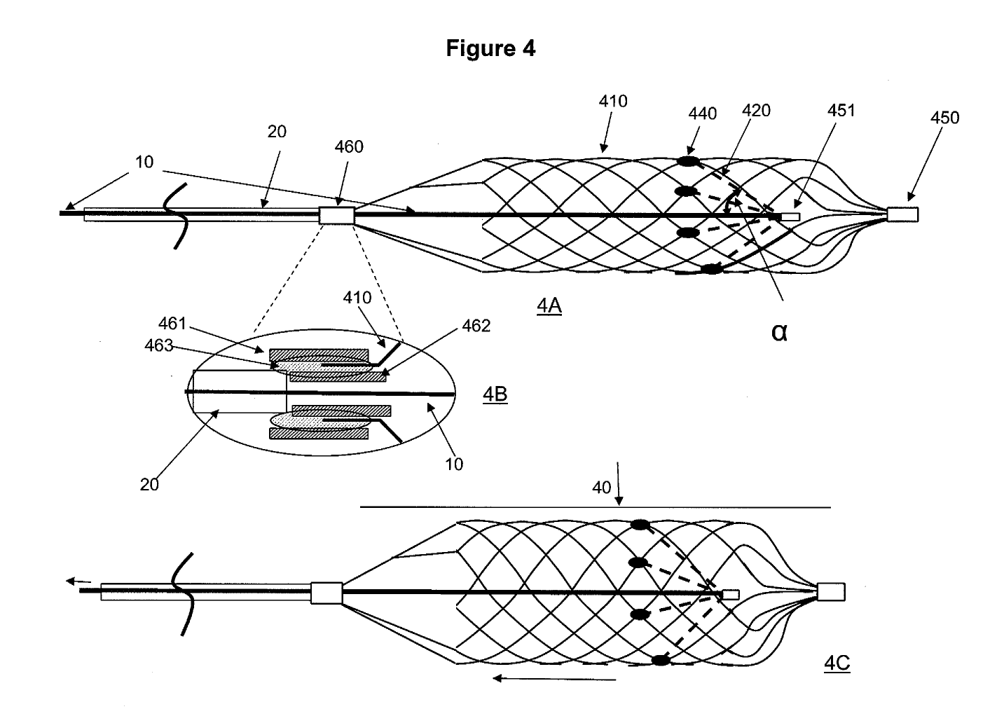

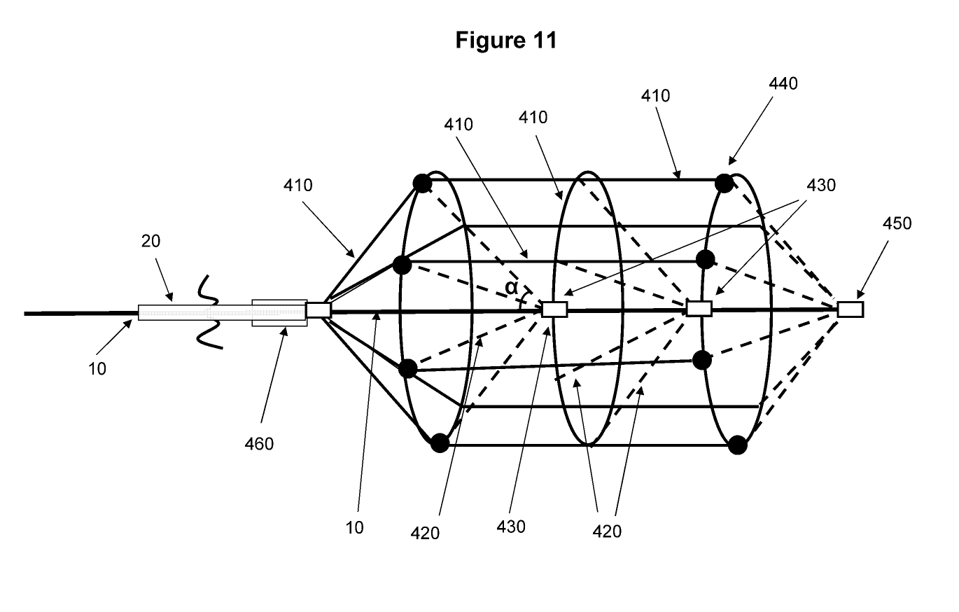

The term “expandable compartment” generally includes a structure that can be inserted into a body lumen to recanalize the blocked vessel or counteract localized flow constriction either by opening the vessel or removing the occlusion. Reconfigurable element is one component to form an expandable compartment. In some embodiments, the reconfigurable element may comprise struts made from tubing or sheet materials (see example in FIG. 3). In some other embodiments, the reconfigurable element may comprise a plurality of wires which can be formed into a mesh (see example in FIG. 4). In some other embodiments, the plurality of wires of the reconfigurable element may be aligned together and form a tubular shape (see example in FIG. 11). The reconfigurable element can be made of metal materials. Some non-limiting examples of such metal materials for the reconfigurable element include nickel-titanium (NiTi) alloy, stainless steel, titanium and its alloys, and cobalt chrome (CoCr) alloys. Alternatively, any polymers or plastics which have desired properties of being reconfigurable element can be used as materials of reconfigurable element production. In further alternative examples, the reconfigurable element can be constructed using two or more different materials.

In some embodiments, a diameter of the struts used in the reconfigurable element may vary from approximately 0.0005 inch to 0.1 inch (12.5 μm to 2500 μm). In some other embodiments, a diameter of the wire used in the reconfigurable element may vary from approximately 0.0005 inch to 0.1 inch (12.5 μm to 2500 μm). The reconfigurable elements are in general flexible and with elastic or super-elastic property. Thus the reconfigurable element's configurations can be reconfigurable. The reconfigurable element, typically comprise at least three different configurations which are referred to a “collapsed (i.e. axially extended, folded or closed)” configuration, “relaxed (i.e. unfolded or open)” configuration, and an “expanded (i.e. radially extended or radially expanded)” configuration. The complete collapsed configuration of the reconfigurable element generally represents a status in which the outer radius of the reconfigurable element becomes minimized while its axial length is maximized. When the device is in its introducer sheath or in a microcatheter, the reconfigurable element is in its collapsed configuration. When the reconfigurable element is pushed out of microcatheter or introducer sheath and if there is no compressive force, i.e. without any constraint, the reconfigurable element is in its relaxed status. The complete expanded configuration of the reconfigurable element generally represents a status in which the outer radius of the reconfigurable element becomes further expanded or maximized. The configurations of the reconfigurable element may be controlled by the control element and supportive element from its complete collapsed status, or relaxed status to expanded status. The outer diameter may vary as the reconfigurable element's configuration changes and could range from approximately 0.01 inch to 0.5 inches (0.25 mm to 12.5 mm) in the collapsed configuration. The expanded configuration diameter may range from approximately 0.04 inches to 1.0 inches (1.0 mm to 25 mm). An axial length of the reconfigurable element may also vary as its configuration changes. In certain embodiments, the axial length of the reconfigurable element may be increased as it becomes collapsed. On the contrary, the axial length of the reconfigurable element may be reduced as it becomes more expanded. The axial length of the reconfigurable element could range from approximately 0.1 inch to 3 inches (2.5 mm to 75 mm).

In accordance with some embodiments of the invention, the supportive element comprises a plurality of wires or struts. The plurality of wires or struts of the supportive element may be in a generally linear form or in a generally non-linear form. In certain embodiments, the supportive element is in a form of wire mesh. In other embodiments, the supportive element is in a braid form. In other embodiments, the supportive element is in a meshed tubular form manufactured from a tube though laser-cutting. In other embodiments, the supportive element is in a meshed sheet form, manufactured through laser cutting or photo etching process. The supportive element is generally configured to adjust a configuration of the reconfigurable element, thereby providing delicate control over the extent of the reconfigurable element's radial expansion. Such delicate control mechanism of the device would be beneficial in many aspects. After delivery and release of the device at the selected treatment site, it may appear that the radius and/or radial force of the self-expanding reconfigurable element may be less than that desired for the application. On such occasions, the supportive element may provide further radial force/pressure to the lumen. It may be desired to occasionally increase or decrease the amount of radial force which the device exerts against surrounding tissue or occlusion. In such cases, the configuration of the reconfigurable element can be dynamically controlled to provide a wider range of radial force in the device according to the present disclosures. The device may also reduce or minimize any unnecessary impact or damage to the blood vessel while the device is being delivered, removed and/or operated. In some embodiments, when the device is delivered, it may provide undesired pressure and/or impact to the lumen when it is released in the vessel and can expand more than the luminal diameter. In such cases, the reconfigurable element's diameter and radial force can be reduced by movement of the control element and supportive element when necessary. In other cases, when the device is being removed the radial force may be too great and potentially cause injury while being pulled back through the blood vessel. Similarly, the reconfigurable element's diameter and radial force can be reduced by movement of the control element and supportive element.

For the purpose of instant illustration, some non-limiting and illustrative examples of the device according to the invention are provided in the following figures. While only few exemplary applications are described herein for the purpose of illustration, many different modifications and alternations, which should be obvious to a person with ordinary skill in the art, can also be done without affecting the scope of the invention. Therefore, not only the examples disclosed in this application but also such obvious modifications and alterations should also be included in the scope of the invention.

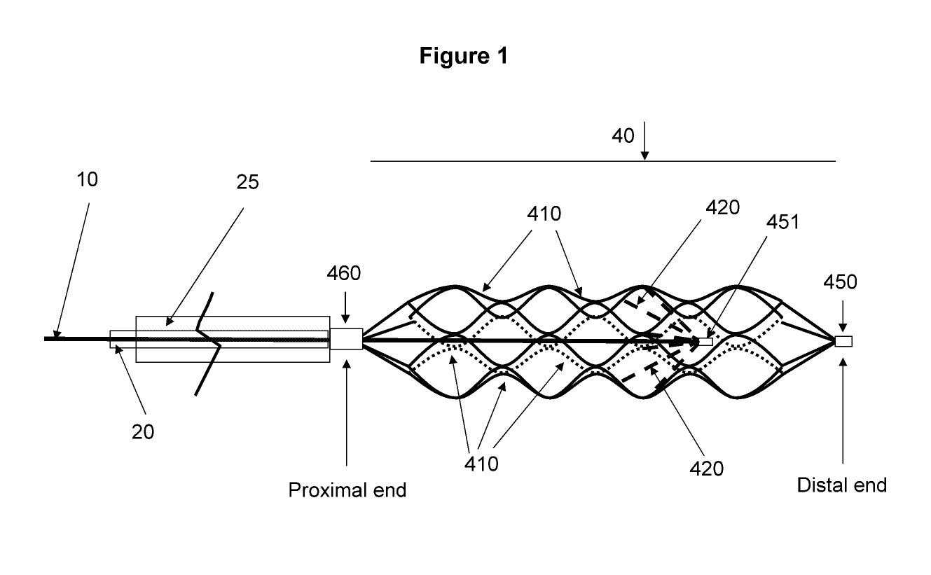

Referring to FIG. 1, a device comprising a reconfigurable element (410), a supportive element (420), a control element (10), and a pusher tube (20) is depicted. The expandable compartment (40) can be present inside the introducer sheath (25).

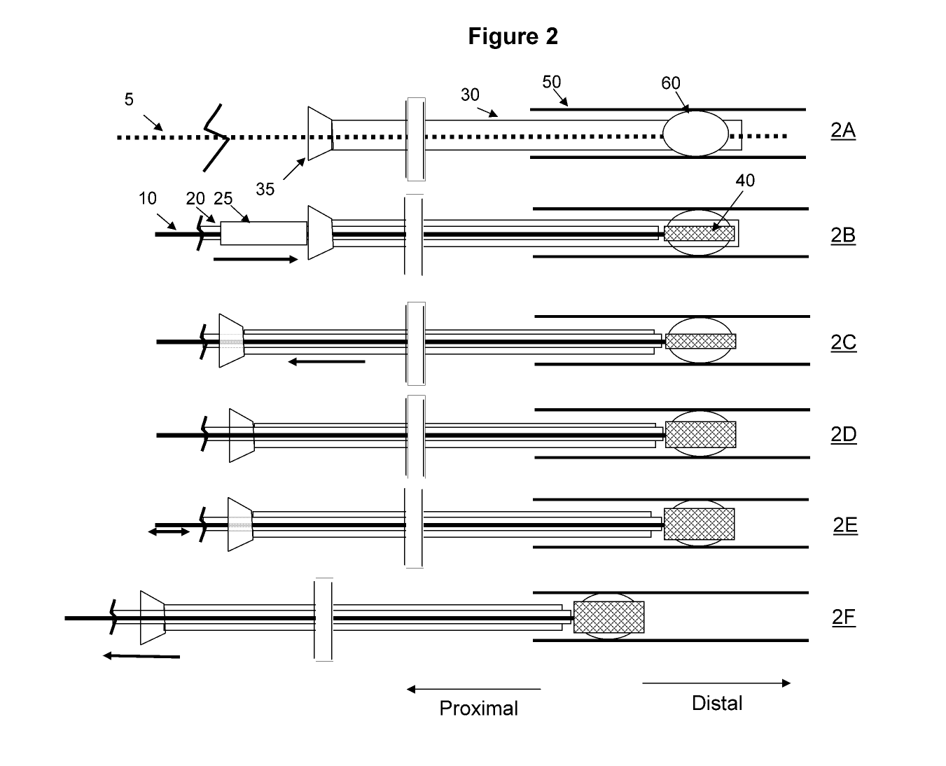

During a clinical procedure, the device can be pushed into a microcatheter (30) via introducer sheath (25) and be further pushed to the lesion site as seen in FIG. 2. The reconfigurable element (410) can be connected to a pusher tube (20), in this case, a thin hollow tube. The supportive element may be associated with the control element. At least part of the control element (10) may be surrounded by a pusher tube (20) and the control element (10) may freely slide through the pusher tube (20). Thus, the movements of the control element (10) and the pusher tube (20) may not be constrained by each other and each can slide freely along the axial axis of the device, as shown in FIG. 3B and FIG. 4B. The microcatheter may be placed at an occlusion location with the help of a guide wire. The pusher tube can be used to push the device into micro-catheter. The expandable compartment of the device can then be pushed out of the microcatheter during treatment. The radial force and the diameter of the reconfigurable element can be adjusted by pulling or pushing the control element proximally or distally.

FIG. 2 illustrates the steps that may need to remove an occlusion from the blood vessel. Accordingly the luminal surface (50) may represent a blood vessel wall. In some embodiments, the device may be delivered to the luminal area (50) in which the occlusion/clot (60) is present. During the delivery, with the help of a guide wire (5), the distal tip of the microcatheter may be navigated to the blocked site and pass through the occlusion (60), as illustrated in FIG. 2A. Angiographic guidance may be used to locate the position of the micro-catheter relative to the vessel and occlusion.

After removing the guide wire (5), the retrieval device can then be introduced into the microcatheter (30). The expendable compartment (40) may be pushed through the microcatheter (30) until the distal end of the expendable compartment (40) reaches to the distal end of the microcatheter (FIG. 2B). As depicted in FIG. 2C, the microcatheter (30) may be withdrawn slightly by pulling it proximally while the pusher tube (20) is held stable. The expandable compartment (40) may be exposed to the clot (60) and partially opened as shown in FIG. 2D. The operator may adjust the configuration of the reconfigurable element (410), i.e. radial force, diameter, and axial length of the reconfigurable element) to allow the reconfigurable element (410) to break or engage the occlusion (60), disrupt at least part of the occlusion (60) and/or expand the lumen of the vessel. Such adjustment of the reconfigurable element's configuration may be achieved at least by pulling or pushing the control element (10) distally or proximally as seen in FIG. 2E. After the occlusion (60) is engaged by the expandable compartment (40), the device, along with the microcatheter (30), may be retrieved from the blood vessel as shown in FIG. 2F.

Alternatively, the micro-catheter can be first placed beyond the occlusion and the expandable component can be opened distal to the occlusion. After maneuvering and adjusting the reconfigurable element's diameter and radial force, the device can be pulled proximally, and the clot can be entrapped by the expandable compartment and pulled out of vessel.

While the embodiments of FIG. 2 illustrate substantial removal of occlusion from the blocking site of the lumen, alternative treatment such as disruption of at least part of the occlusion can be used. The occlusion is often substantially soft and may be broken up with a relatively minor impact. In such cases, the reconfigurable element may in part break up the occlusion into smaller components. The pieces of the occluding material can be collected and removed from the body with the device. If the reconfigurable element's wires or struts cut through the occlusion (e.g. blood clot) while the reconfigurable element expands radially in the blood vessel, portions of the clot become contained in the expandable compartment as it expands to the full diameter of vessel. By slightly collapsing the reconfigurable element through pulling it partially into the micro-catheter tip, the openings between the wires/struts of the reconfigurable element cells would be made smaller. This may maintain the clot in the expandable compartment. The supportive component may also help to maintain the clot within the expandable compartment. The whole device containing the portions of the clot can then be pulled out of the blood vessel. In further alternative embodiments, the device may dilate/expand the lumen so that the flow can be re-established at the blocking site of the lumen. It is also possible that the occlusion may not be soft enough to allow the device substantially engage the occlusion. In such cases, the configuration of the reconfigurable element may be controlled to engage at least part of the occlusion and mobilize the same. When the reconfigurable element's diameter is open fully, its wires or struts will push against the clot. While pulling the proximally, the friction between the reconfigurable element and the clot may cause clot to become dislodged from the vessel wall and removed.

FIGS. 3 and 4 shows devices according to some embodiments of the invention. The device comprises a pusher tube (20) which may be connected with proximal end connector (460) of the reconfigurable element (410). The pusher tube as well as the control element may be substantially long. In some embodiments, the pusher tube and the control element may long enough for an operator to control the retrieval device from outside a body via the pusher tube and the control element. In some embodiments, the pusher tube and the control element may extend about 100 cm, 110 cm, 120 cm, 130 cm, 140 cm, 150 cm, 160 cm, 170 cm, 175 cm, 180 cm, 185 cm, 190 cm, or 200 cm. These wires can be extended over 200 cm, if necessary.

The reconfigurable element (410) may comprise at least two ends, a distal end and a proximal end. The distal end of the reconfigurable element (410) generally refers to an end that may enter the body prior to the proximal end. The proximal end of the reconfigurable element (410) generally refers to an end in which the reconfigurable element (410) is associated with the pusher tube (20).

In some embodiments, the distal end of the reconfigurable element (410) is closed, which means that the distal ends of the reconfigurable element wires or struts (410) are held together by means of, for example, welding, soldering, or gluing, with or without a connector (430 or 450). In some embodiments, the distal end of the control element (10) and the distal end of the supportive element (420) may be held together with or without connector (451) via a means of, for example, welding, soldering, or gluing etc. Alternatively, a distal end connector (451) may be used to couple the distal tip of control element (10) and the distal tip of supportive element (420). At the proximal end of the reconfigurable element (410), there is an outer proximal end connector (461) and an inner proximal end connector (462), both of which may be tubular structures as seen in FIG. 3B-8B. The proximal wire or strut ends of the reconfigurable element (410) may be placed between the inner and outer proximal end connectors (461 and 462) and are fixed in place by means described above. The control element (10) may pass through the lumen of the inner proximal end connector (462) so that it can slide in the proximal end connector (460) freely.

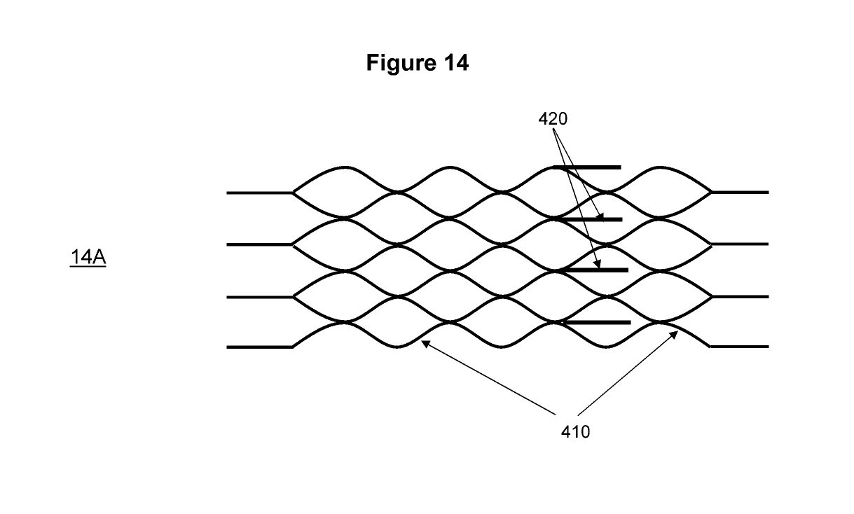

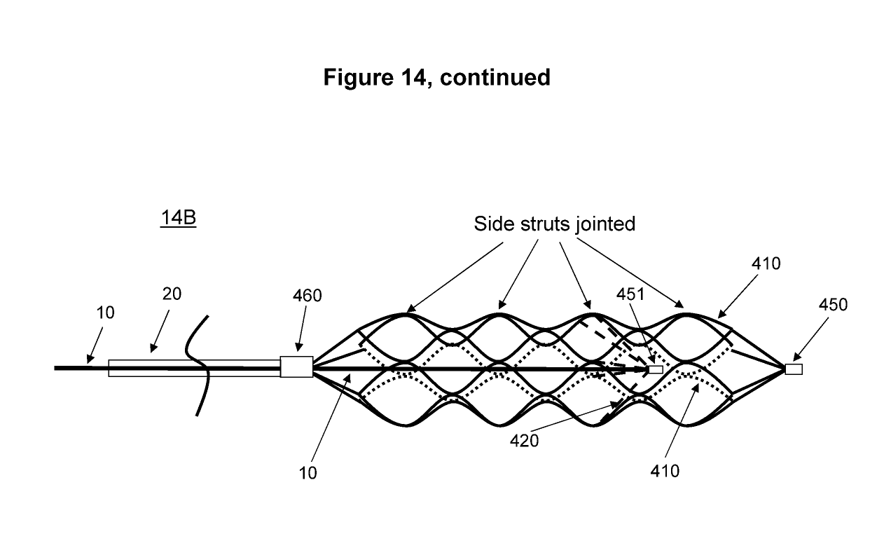

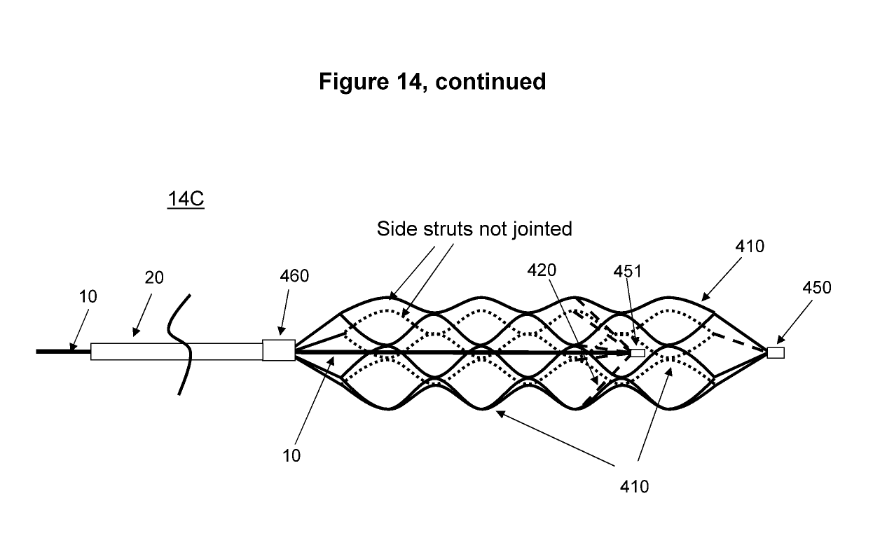

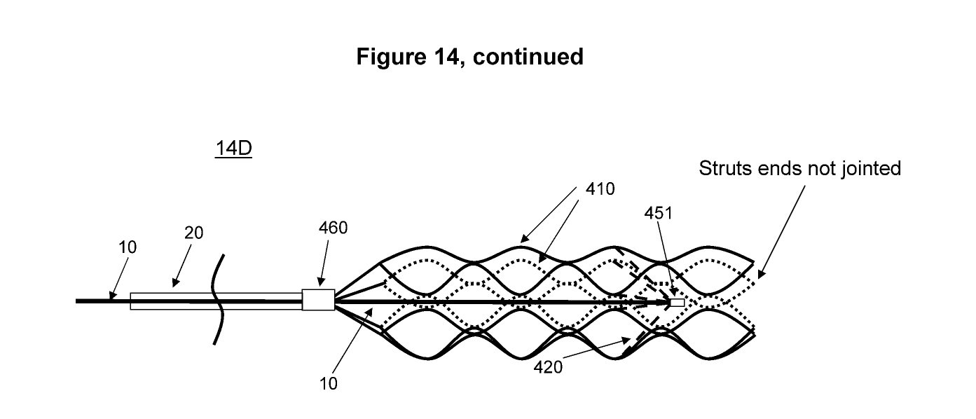

The device further comprises a supportive element (420) which may be associated with the reconfigurable element's wire/strut (410) and the control element (10). The supportive element (420) may be associated with the control element (10) via a connector (430 and/or 451), for example, as seen in FIG. 7. In some embodiments, the supportive element (420) is associated with the distal end of the control element (10) in a substantially immobilized manner. Accordingly, as the control element (10) is pulled proximally, the distal end of the supporting element (420) may be pulled proximally. The supportive element (420) may be attached to the reconfigurable element (410) as shown in FIG. 3 and FIG. 4. In this embodiment the supportive element ends that are attached to the reconfigurable element (410) may move outward causing the reconfigurable element (410) to enlarge (as shown in FIG. 3C and FIG. 4C). This will increase the radial force of the reconfigurable element. In other embodiments the supportive element (420) is made from the same piece of material as the reconfigurable element (410) as shown in FIG. 3 and FIG. 14. Thus, jointing these two components is not needed. In this embodiment pulling the control element (10) proximally will result in enlargement of angle α (FIG. 3C) between the supportive element (420) and the control element (10) causing the reconfigurable element (410) to become enlarged (as shown in FIG. 3D). This will increase the radial force of the reconfigurable element.

As further depicted in FIGS. 3B and 4B, the control element (10) may be present in the lumen of the proximal inner connector (462) and slide freely through the connector (460). In some embodiments, markers (440) may be added to the device, for example, at which the supportive element (420) is associated with the wire/strut (410). Such markers may include radiopaque materials which help visualization/monitoring the position and or movement of the device in the body. Some non-limiting examples of radiopaque markers may comprise gold and/or platinum. The markers may be added at some of the attachment points between the supportive element (420) and any portion of the wire/strut of the reconfigurable element (410), control element, or supportive element (420), if desired. For example, the markers may be applied at about the distal and/or proximal ends of the reconfigurable element or be coated on any part of the wires and/or connectors. The radiopaque property of the device can also be achieved by using CoCr alloy as the reconfigurable element, control element, and, or supportive element.

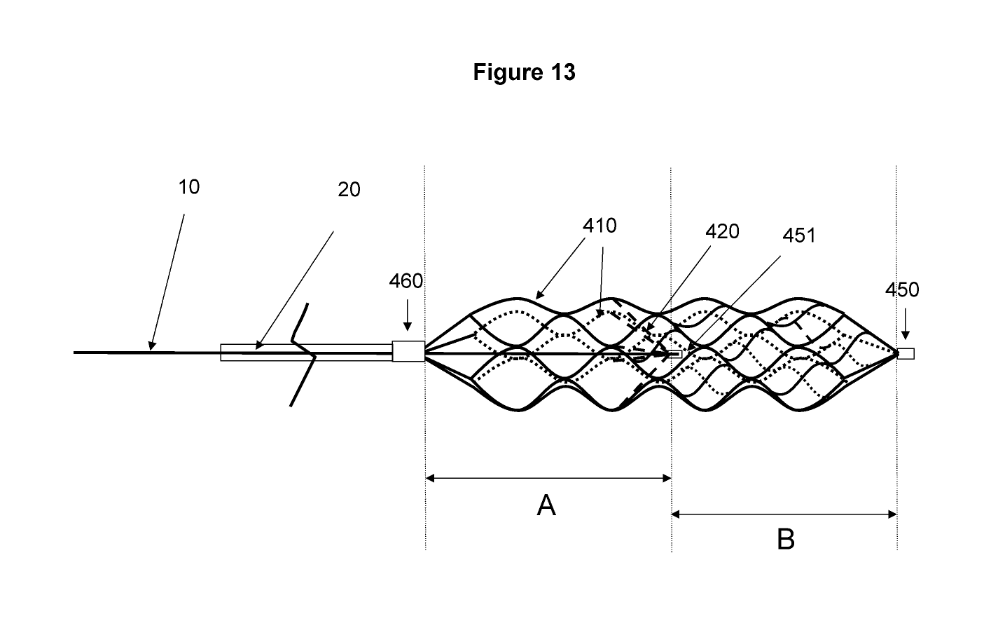

The supportive element can be constructed in a variety of forms. The particular example shown in FIG. 3, FIG. 13 and FIG. 14 is a build-in structure, i.e. both the supportive element and reconfigurable element are built from one piece of material, either from tubing or flat sheet. Thus additional joining/bonding between them is not needed.

FIG. 4 comprises a plurality of supportive element that is in a form of substantially linear wire. Materials used to manufacture supportive elements would be metal or non-metal materials. Some non-limiting materials used for the supportive element include, but not limited to, nickel, titanium, NiTi, stainless steel, cobalt chrome and any alloys of the foregoing. The configuration of the reconfigurable element may be controlled by movement of the control element and the supportive element. After the microcatheter is passed through occlusion, the device may be delivered into the microcatheter. When the device is unsheathed from the microcatheter (See, for example, FIG. 2), it can be exposed and engage with the occlusion.

In some embodiments, the reconfigurable element may be self-expanding once it is out of the microcatheter and its configuration can be further altered by pulling or pushing the control element distally or proximally. The radial force of the reconfigurable element can be further controlled via the supportive element and the control element. If the self-expandable compartment is configured to expand more than the diameter of the blood vessel, this self-expansion process may exert too great a force on the wall. This may result in injury to the vessel leading to tearing or perforation. Accordingly, it would be beneficial to be able to control the configuration of the reconfigurable element (including a radial force, size and shape of the reconfigurable element) in a delicate manner to achieve safe and efficient treatment. In addition, the reconfigurable element may need to be further expanded or the radial force of the reconfigurable element increased after achieving its nominal self-expanded diameter. For example, the radial force of the reconfigurable element may need to be enhanced to substantially engage, cutting through, and/or mobilize the occlusion. Therefore, it is expected that the configuration of the reconfigurable element would need to be dynamically changed during the treatment procedure. The device according to at least some embodiments of the invention is designed to provide such dynamic control of the reconfigurable element.

Alternatively, the reconfigurable element may not be self-expanding and thus the entire opening and closing of the reconfigurable element may need to be controlled. In such a case, when the expandable compartment is unsheathed from the microcatheter to treat the condition in the lumen, the control element may be slightly pulled proximally so that the reconfigurable element may be axially expanded. Similarly to the self-expanding reconfigurable element, further expansion or collapse of this non self-expanding reconfigurable element would be controlled by movement of the control element attached to the supportive element.

In some embodiments, the device comprises at least two mechanisms to control the configuration of the reconfigurable element. First, there is a control provided from the supportive element. The supportive element is generally controlled by the control element. Upon the distal movement of the control element, the supportive element also becomes extended along the axial axis. When the control element retreats proximally, the supportive element would become radially expanded, which would provide further supporting pressure to the reconfigurable element. As the control element moves more proximally, the supportive element would become more expanded and thus the angle between the control element and the supportive element (shown as α in FIGS. 3 and 4) would be increased. The degree α may range between about 0 to 90 degree. In addition, the configuration of the reconfigurable element (e.g. the overall shape, axial length and outer diameter of the reconfigurable element would be controlled by movement of the control element. Distal movement of the control element would cause the reconfigurable element to shift to its collapsed status, i.e. the reconfigurable element becomes axially extended and its outer radius is reduced while the axial length is increased. Proximal movement of the control element would shift the configuration of the reconfigurable element toward its expanded status, i.e. the reconfigurable element becomes radially expanded. As a result, the axial length of the reconfigurable element may be reduced while the outer diameter is increased. This shift of the reconfigurable element toward its expanded status would enhance the radial force of the reconfigurable element.

Connectors can be metal hypo-tubing, such as stainless steel (SS), Platinum, Gold, Nitinol tubing, plastic tubing such as Polyimide tubing, or can be a segment of coil made from SS, Platinum alloy, Gold, or CoCr alloy wires etc. When using radio opaque material such as Gold, Platinum etc, the connector can also serve as marker.

The configuration of the reconfigurable element would be controlled in the blood vessel in order to remove an occlusion from the lumen and/or expand the lumen in some embodiments. Once the occlusion is engaged by the reconfigurable element, the device would be withdrawn from the lumen and eventually from the body. When the device is withdrawn from the lumen, the expandable compartment that is engaged with the occlusion may be partially withdrawn back into the microcatheter or left distal to the microcatheter. The expandable compartment with clot and microcatheter can be simultaneously pulled back into a guiding catheter that has larger inner diameter. The relative position among the micro-catheter, guiding catheter, and the expandable compartment can be determined using fluoroscopy.

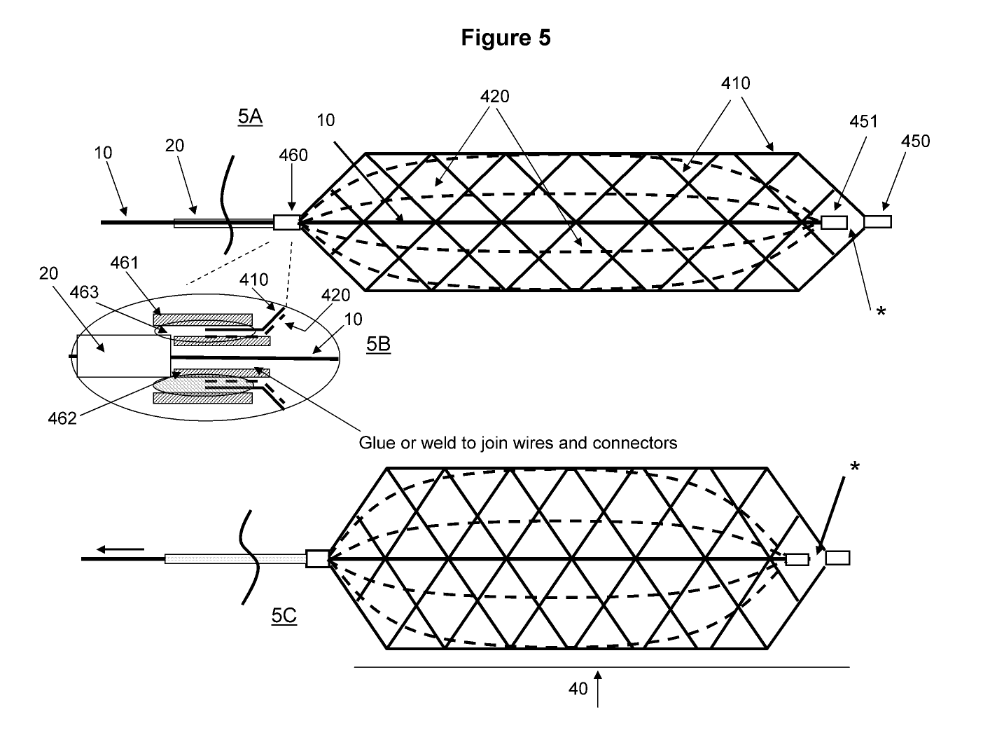

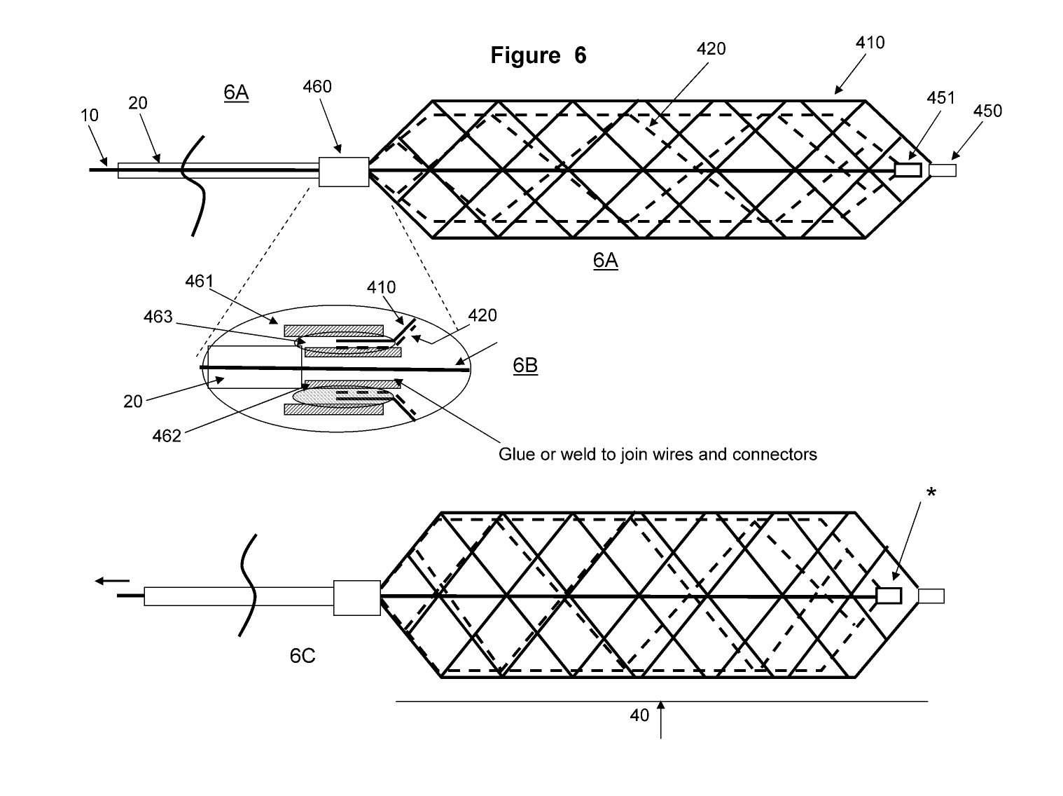

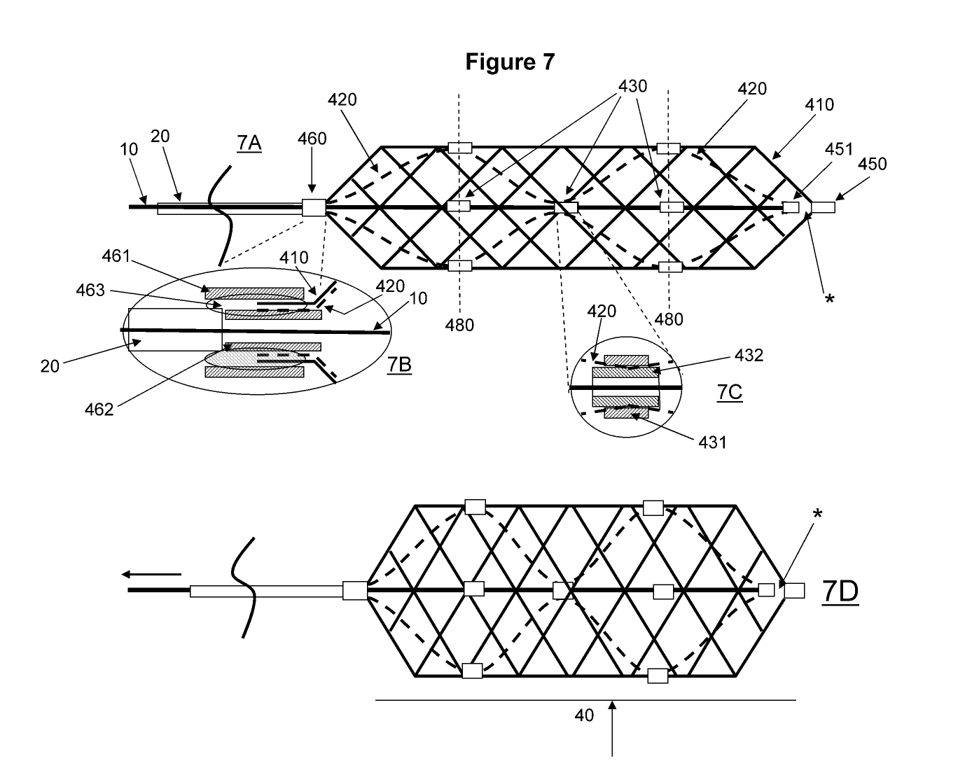

An alternative embodiment of the device is provided in FIGS. 5 and 6. In this particular embodiment, the supportive element (420) comprises a plurality of wires as shown in FIG. 5, or braid structure as shown in FIG. 6. The supportive element (420) extends from the proximal end of the reconfigurable element or wire/strut (410) and ends before the distal end of the reconfigurable element (410) (as marked with “*” in FIGS. 5-7), thereby forming a double-layered expandable compartment. The distal end of the supportive element (420) may be fixed with the distal tip of control element (10).

As seen in FIGS. 5B and 6B, association of the reconfigurable element (410) with the supportive element (420) may be done via a proximal connector (460). The proximal connector may comprise at least two compartments, an outer proximal connector (461) and an inner proximal connector (462). The proximal ends of the reconfigurable element wire and the proximal ends of the supporting element are fixed with adhesive, welding, soldering (463), or through mechanical joint in between proximal outer and inner connectors. In some other embodiments, these inner and outer proximal connectors may be a tubular or coil structure and the control element may freely slide through the inner connector.

In the above device, when the control element (10) retreats proximally, it may cause expansion of the supportive element (420), providing support to the reconfigurable element wire/strut, which leads to enlargement of the diameter and enhancement of the radial force of the reconfigurable element (410).

A further alternative embodiment of the device is provided in FIG. 7. In this particular example, the supportive element (420) is configured to comprise two plateau positions (480) in which the supportive element (420) may provide the maximal strength of support to the reconfigurable element wire/strut (410). The supportive element (420) may be formed into a sinusoidal shape as seen in FIG. 7. The distal end of the supporting element is fixed to the control element (10). Both the proximal ends of the reconfigurable element and supporting element are fixed to the proximal connectors as shown in FIG. 7B. As shown in FIG. 7C, the middle (thin) section of the supporting element is connected via middle outer and inner tubing connecters, with control element moving freely inside the proximal and middle connectors (430).

One advantage of having two or more plateaus in the supportive element is that the radial force can be selectively enhanced at preferred positions. As readily seen in FIG. 7, the reconfigurable element (410) would receive the maximum strength of support from the supportive element (420) at two plateau positions (480) and the strength of the support would reduce as it is distant from the plateaus. Accordingly, the device can provide a wider range of radial force to the luminal area if desired. Moreover, the number of supportive element can also vary from two to more which may be circumferentially distributed around the control element (10) to vary the radial force and/or the outer shape and density of the device.

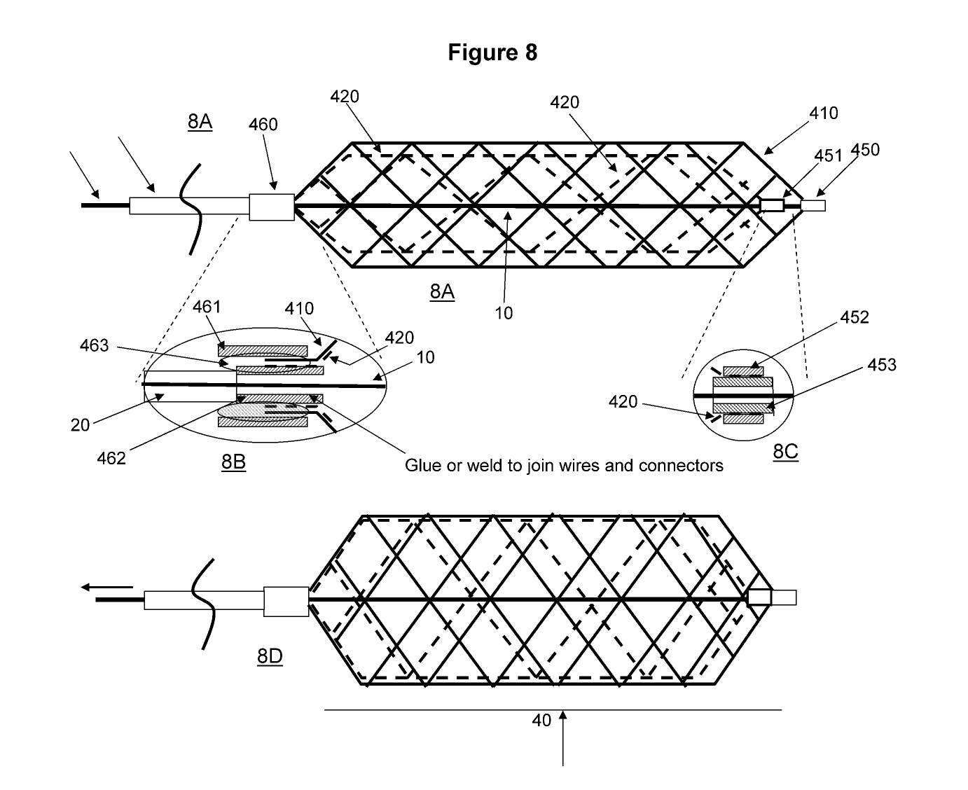

A still further alternative embodiment of the device is provided in FIG. 8. In this particular example, the control element (10) runs through the distal end of the supportive element (420) and reaches to the distal end of the reconfigurable element (410). The distal end of the supportive element may freely slide along the control element as seen in FIG. 8C. The supportive element (420) may be associated with the control element (10), for example, via a connector (451) at the distal end of the supportive element (420). The connector 450 comprises of a supportive distal inner connector (453) and supportive distal outer connecter (452), joining the distal end of the supportive elements in between them. The control element (10) can slide in the lumen of the inner connector. When the control element (10) is pulled proximally, the distance between the connector (450) of the reconfigurable element and the distal end of the supportive element (451) become shorter. When the two connecters are in contact to each other, the supportive element will be expanded, pushing against the reconfigurable element, and generating additional radial force as seen in FIG. 8D. One benefit of this particular embodiment of FIG. 8 would be that the distal end (451) of the supportive element and the distal end (450) of the reconfigurable element are aligned to the axial direction, avoiding tilting of the supportive element tip while the control element is pulled proximally. It may also avoid constraint in the wire axial length between the reconfigurable element (410) and the supportive element (420) when the device is retracted into the microcatheter.

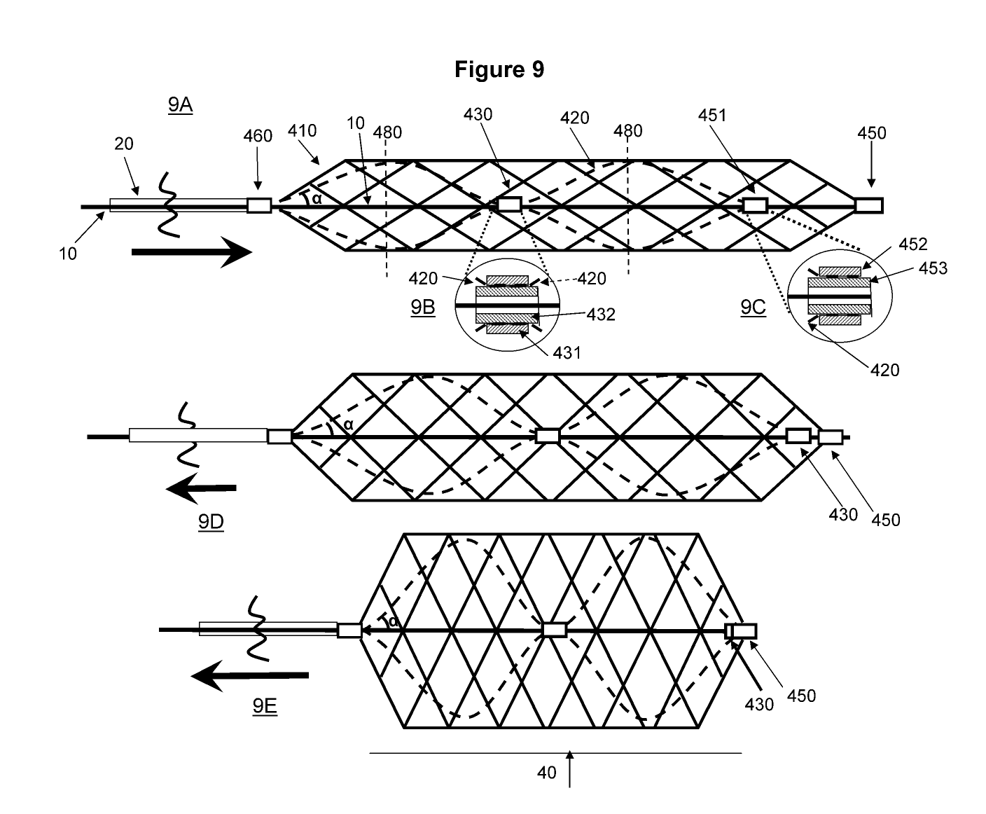

A still further alternative embodiment of the device is provided in FIG. 9. In this particular example, the supportive element (420) comprises at least two plateau positions (480). Moreover, the distal end of the supportive element (420) may not be substantially immobilized at about the distal end of the device. Therefore, the supportive element (420) may be associated with the control element (10), for example, via connectors (430) at the distal tip of the supportive element. These connectors (430) may be configured to freely slide along the control element. The structure of the connector (430) also comprise of inner and outer connectors to ensure control element can move freely through the connectors. Accordingly, when the control element is pulled proximally, the reconfigurable element distal connector (450) may move closer to the distal end of the supporting element. When the two connecters are contacted to each other, the supporting element will be expanded, and will push against the reconfigurable element (410), generating additional radial force as seen in FIG. 9E,

This particular device of FIG. 9 may provide at least three benefits. The supportive element comprising more than one plateau position which may allow the radial force to be selectively enhanced at preferred positions. Accordingly, the device can provide a wider range of radial force to the luminal area if desired. In addition, similar to the device of FIG. 8, the distal end of the supporting element and the distal end of the supporting element is aligned to the axial direction by the control element, avoiding tilting of the tips while the control element is pulled proximally. It can also avoid constraint in the wire axial length between the reconfigurable element (410) and the supportive element (420) when the device is retracted into the microcatheter.

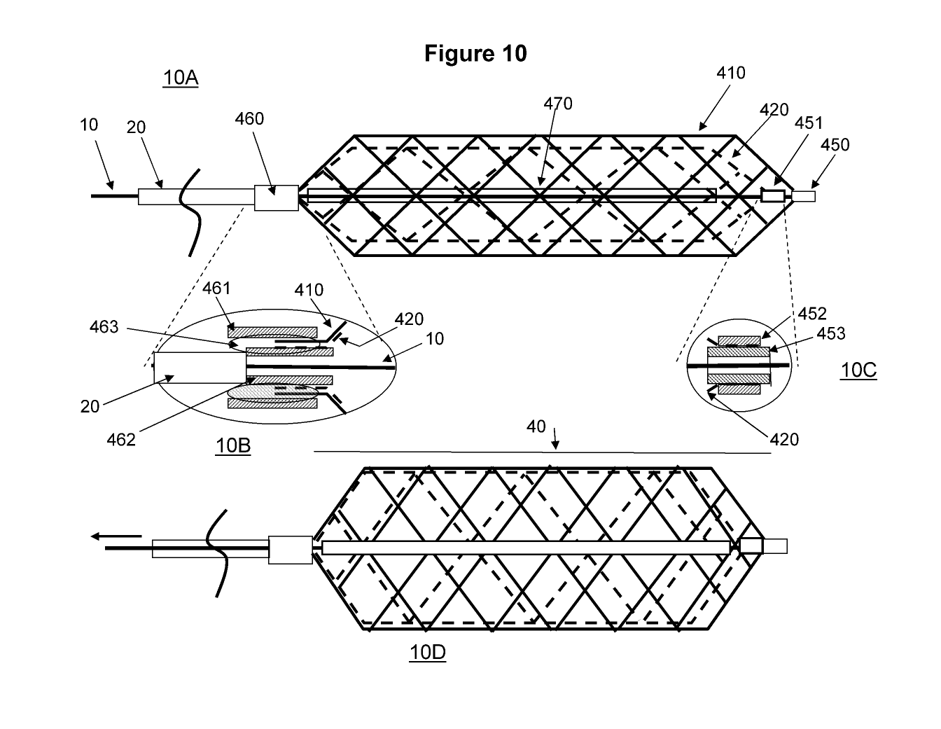

According to some embodiments of the invention, an adjustment tube may be utilized in the device in all the described designs. As seen in FIG. 10, the adjustment tube (470) may be placed over the control element between the proximal and distal ends of the supportive element. The adjustment tube may optionally be freely slide along the control element. While the adjustment tube is present in the device, it may prevent the connector (451 or 430) to be too close to the proximal ends of the expandable compartment. Accordingly, the device with the adjustment tube (470) may prevent excess axial expansion of the reconfigurable element. The tubing can also prevent or reduce friction between the control element and the supportive element struts/wires when pulling the device back into an introducer sheath or a micro-catheter.

A still further alternative embodiment of the device is provided in FIG. 11. In this particular umbrella-shaped device, the reconfigurable element (410) may comprise a plurality of wires and form a tubular structure as seen in the figure. The supportive element, which may also comprise a plurality of wires, can be used to alter the configuration of the reconfigurable element. The supportive element (420) may be associated with the control element (10) as well as the reconfigurable elements (410) and at least some of the association positions may be coupled with markers (440). The supportive element (420) as well as the reconfigurable element (410) may be associated with the control element (10) in a substantially immobilized manner. All wires of the supportive element (420) may be associated with the control element (10) via a connector (430). The connectors (430) are fixed to the control element. Therefore, pulling the control element (10) proximally or pushing it distally may also act on the other wires (i.e. the reconfigurable element and the supportive element) accordingly.

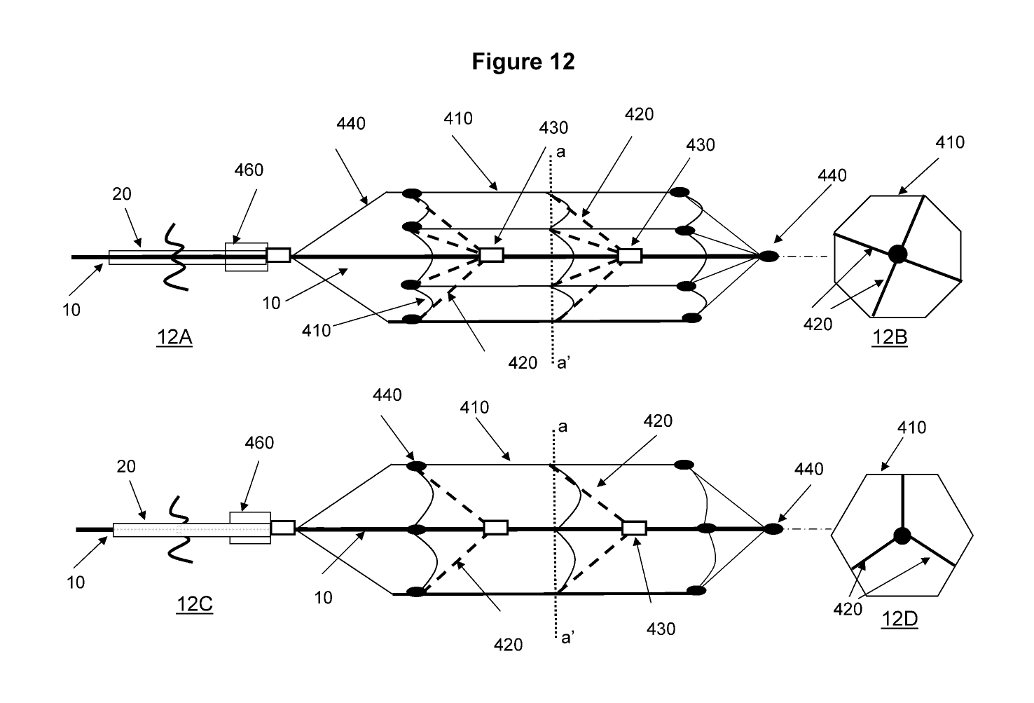

Some non-limiting and illustrative alterations of the foregoing device are shown in FIG. 12. In the device depicted in FIGS. 12A and B, the reconfigurable element (410) comprises 8 generally linear wires aligned along the axial axis of the device. The device further comprises 2 sets of the supportive element (420) distributed between the proximal and distal ends of the device. Each of this set of the supportive element (42) may comprise 4 wires to manipulate the radial force of the device. Alternatively, the device of FIG. 12C comprises the reconfigurable element (410) comprising 6 generally linear wires aligned axially and the supportive element (420) comprising 3 wires in each set. In addition, any further and other alterations such as using 1 or 2 set of the supportive element as well as using more than 3 sets of the supportive element can be applied to the device. Moreover, the reconfigurable element and the supportive element may be in a form of wire mesh (braid) similar to those seen in FIG. 7, if desired.

Another embodiment according to the present invention is illustrated in FIG. 13. In this particular example, the size of reconfigurable element cell and strut size (e.g. thickness of wire) can be varied within a single device. From the proximal to distal end of the reconfigurable element, the cell may change from large size to small size, and the strut size may change from thick to thin strut. The device illustrated in this figure, the zone A has generally larger cells in the reconfigurable element (410) than those in the zone B, or vice versa. Further, the strut size (or thickness) can be thicker in the zone A as compared to that of the zone B, or vice versa. Advantages of these embodiments may include at least one or more of the following:

1) The large proximal cell size may have less wire density which can increase the force and pressure each strut exerts when the radial force is increased. This may enable the reconfigurable element (wire or strut) to cut through or break clot more easily. A clot may also fall into the expandable compartment more easily because of wider opening. The small sized distal cell is to catch and hold the clot debris that are broken from the proximal end of the expandable compartment, so debris would not escape from the device and go to down stream.

2) The strut size of the reconfigurable element can also change from the proximal end to distal end, with wide/thick strut at the proximal end and thin struts at the distal end. The large and strong proximal end strut would have grate stiffness and can cut the clot more easily. Due to the increased number of cells at the distal end the strut size at the distal end may need to be thin to enable the device to keep a small profile in its compressed state to be able to fit in a microcatheter.

Accordingly, the examples shown in the application should not be considered to limit the scope of the invention and many different modifications and alternations, which should be obvious to a person with ordinary skill in the art, can also be done without affecting the scope of the invention. Therefore, not only the examples disclosed in this application but also such obvious modifications and alterations should also be included in the scope of the invention.

The device according to some embodiments of the invention can be manufactured by a variety of techniques that are known in the art. For example, the reconfigurable element and the supportive element can be fabricated from the some piece of material by laser-cutting a hypo-tube. The hypo-tube after being cut by a laser may be heat set to a desired shape and size of the reconfigurable and the supportive components, which can be further assemble into a device as illustrated in FIG. 13.

Alternatively, reconfigurable element/struts and supportive elements/struts can be made form the same thin sheet by laser cutting or photo etching as seen in FIG. 14A. The component may be heat set to a desired shape and size of the reconfigurable and supportive components. These components may be further assembled into a device. The sides of the component can either be jointed using adhesive, welding, soldering, and mechanical jointing etc. to form a closed-sided-expandable compartment (see FIG. 14B), or simply left open as open-sided-expendable compartment (FIG. 14C).

The distal end of reconfigurable element can either be close-ended (wires or struts ends are joined, shown in FIGS. 14B and 14C), or can be open-ended (i.e. wires or struts ends are not joined, as shown in FIG. 14D).

Both the hypo-tube and metal sheet may be made of one or more selected from the group consisting of nickel-titanium (NiTi) alloy, stainless steel, titanium (and its alloys), and cobalt chrome (CoCr) alloys etc.

One advantage of many embodiments of the above-listed techniques of processing the hypo-tube or the thin sheet is that association (or joining) of multiple wires (e.g. between the reconfigurable element and the supportive element) may be avoid, so the device profile (size) may be reduced. These embodiments compare favorably to braid wire structures, since the struts of the reconfigurable element are all connected at the corners of each cell unit or window. The radial force can be controlled through cell shape and structure design without increasing the profile of the device.

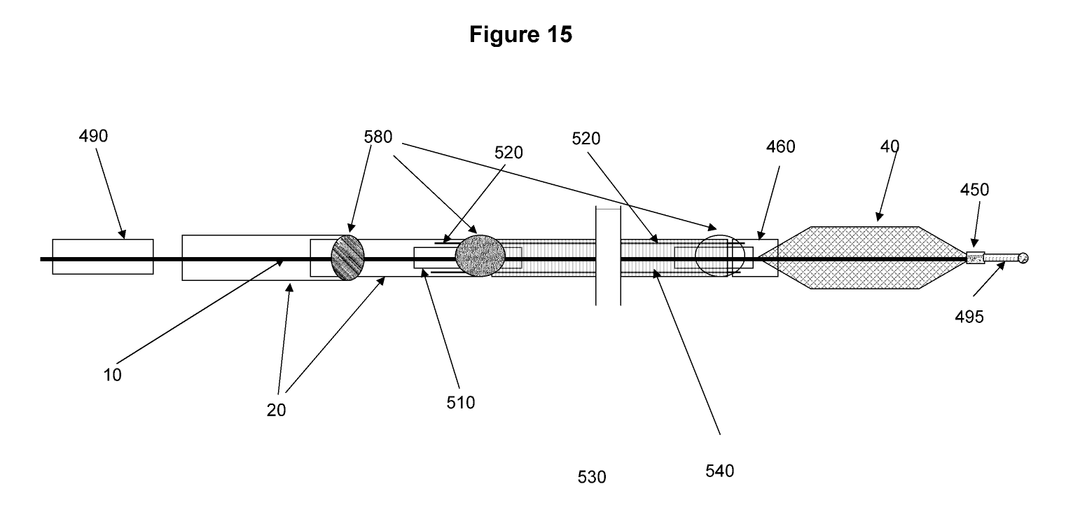

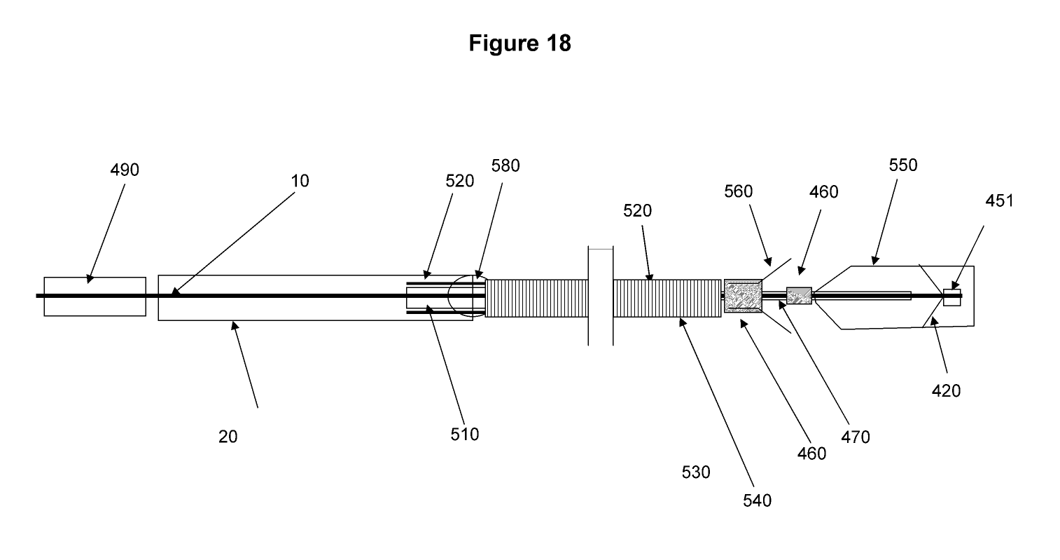

Some embodiments of the present invention relate to a device that is designed to place the expandable compartment in the vasculature of a subject. The subject can be a patient who is in need to treatment such as removing blood clot and/or recovering blood flow in body. An exemplary, non-limiting embodiment of the device is illustrated in FIGS. 15 and 18. According to some aspects, there is a coil section (540) in between the pusher tube (20) and the expandable compartment (40). This coil section can be considered as a part of the pusher tube. The function of the coil (540) can make the distal section of the device flexible, so the device can pass through tortuous vessels. To improve the device pushability, plastic tubing, such as PTFE, PET etc. may be added to outside of the coil, or simply replace the coil as a flexible pushing component at the distal end of the pusher tube. If the coil is used, one or more than two thin wire (520) may be used to link the connector of the expandable compartment (40) and the pusher tube (20) in order to prevent the coil (540) from stretching. According to further some aspects, a pusher tube (20) may be connected to the coil (540). Further, the control element (10) can slide freely, in the lumen of the pusher tube (20), as well as in the coil section and the proximal connecter (460) of the reconfigurable element. According to still some other aspects, at the proximal end of the device, a control element handle tubing (490) may be added and fixed to the proximal end of control element (10). With this feature, an operator can easily grab the control element handle tubing (490) to control the opening or closing of the reconfigurable element. All the connections between parts can be joined through adhesive, welding, soldering, or etc. Referring to FIG. 15, a flexible coil (495) may be added to the distal end of the expandable compartment to make the device tip atraumatic, avoiding poking the vessel lumen.

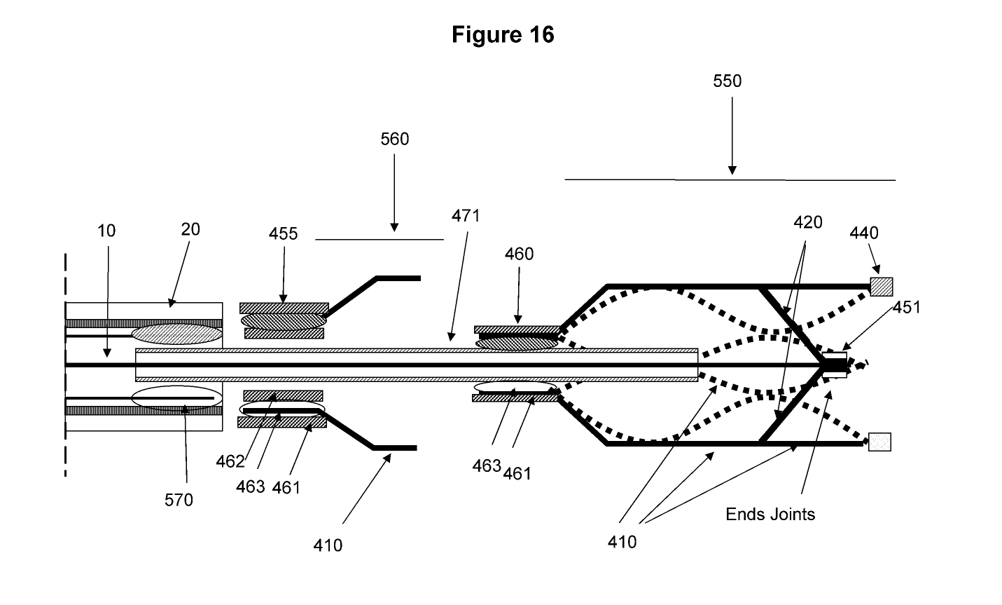

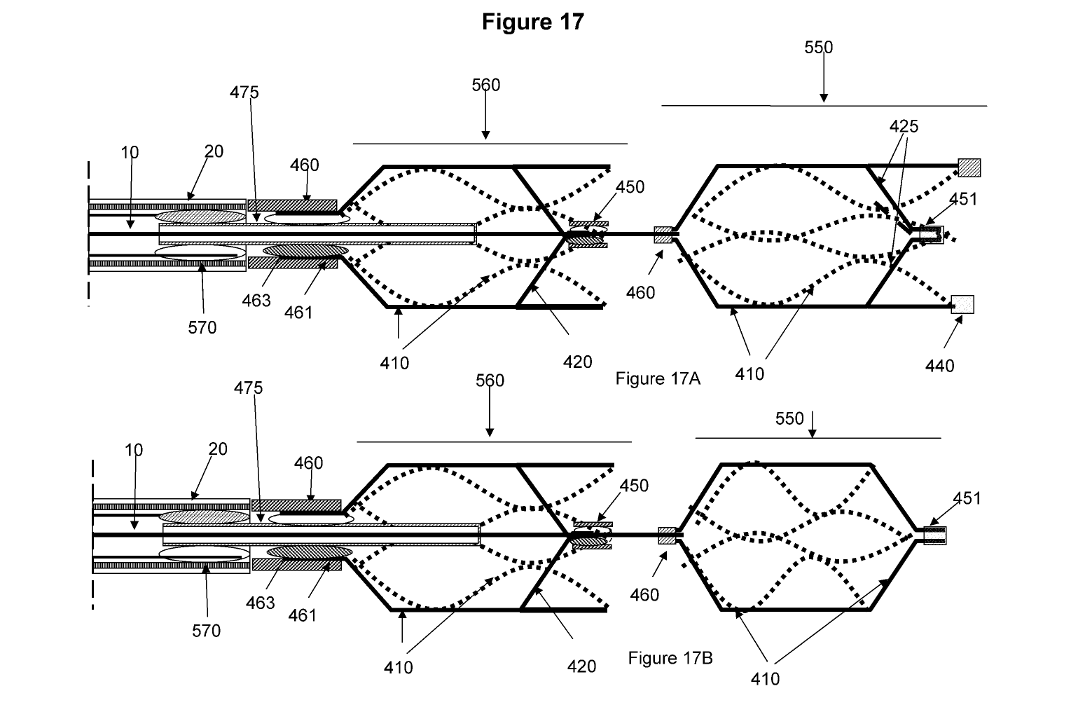

In FIG. 16 and FIG. 17, further alternative embodiments are illustrated. In these embodiments, the device may comprise two structures, a distal expandable structure (550) and a proximal expandable structure (560). For the device shown in FIG. 16, The distance between the two expandable structures can be changed/adjusted, i.e. the distal structure may be pulled/slide toward the proximal structure or pushed/slide away from the proximal structure, for example by pushing and pulling the control element (10).

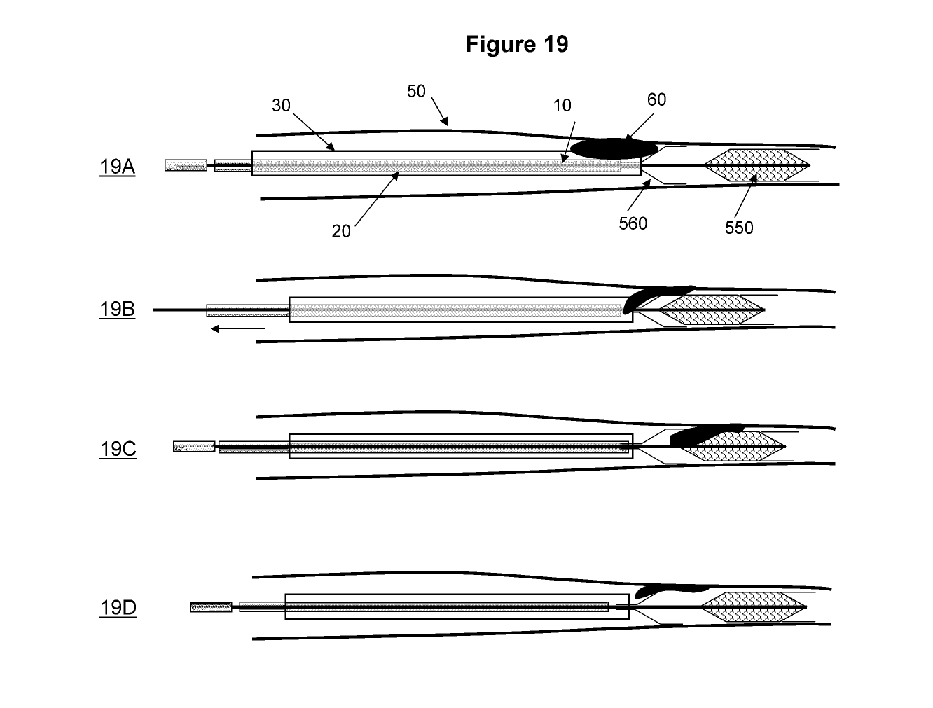

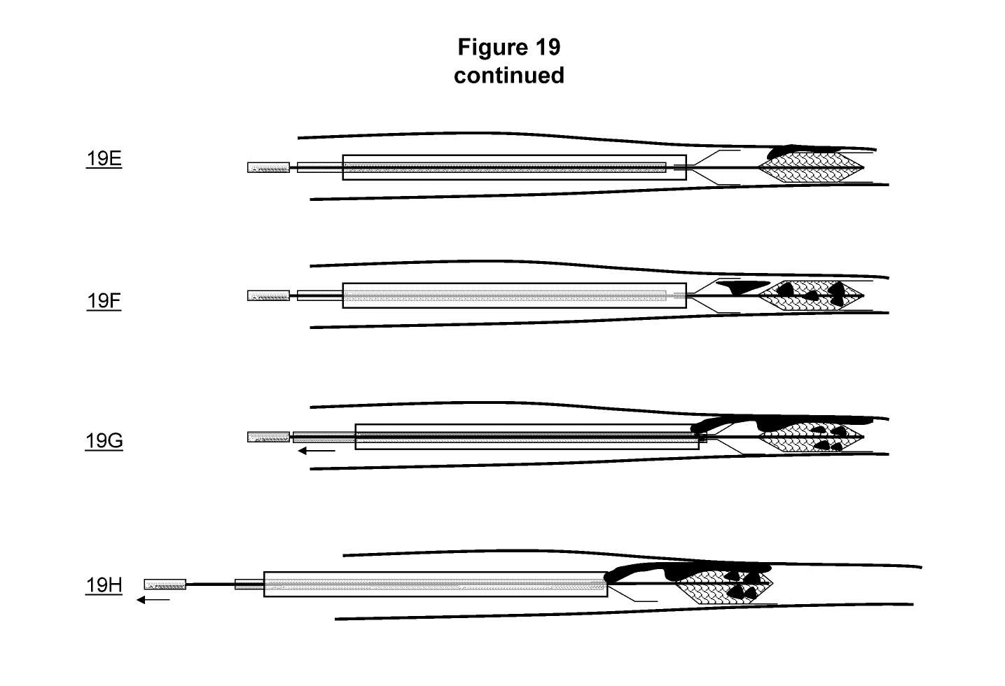

Referring to FIG. 16, the distal structure (550) is in a form of basket or expandable compartment comprising a reconfigurable element (410), a supportive element (420), and a control element. The distal tip of the control element (10) can be connected to the supportive element (420), and move freely inside a sliding tube (471) and pusher tube (20). By adjusting the supportive element through the control element, the radius and radial force of the distal structure can be adjusted. As illustrated elsewhere in the application, adjustment of the supportive element can be achieved by pushing or pulling the central element. In some other embodiments, the proximal end of the sliding tube (471) can be fixed to the distal end of the pusher tube. The distal end of the distal structure (550) can be fixed to a middle point of the sliding tube via a connector (460). The proximal structure may be an umbrella-shaped component (560). Its proximal end may be associated with a connector (455) which may comprise outer and inner connectors. The inner ID of the connector is generally larger than the outer diameter of the sliding tube, so that the sliding tube can slide freely in the connector. During clot retrieving process, while unsheathing the microcatheter, the proximal structure is held by the tip of microcatheter due to friction. It may be separated from the distal structure. When pulling the pusher tubing, the distal structure moves toward the proximal structure. A clot in between the two structure can be engaged (the detailed mechanism of how the device catches clot will be further illustrated in FIG. 19). If pulling the control element proximally, the distal structure may be further expanded. The segment of the sliding tube (between connector 460 and tip of the sliding tube) has the same function as adjustment tube (470) in FIG. 10, i.e. to prevent over expansion of distal structure as illustrated in previous section. Markers (440) can also be added to the distal tip of the distal and proximal structures as needed.

Referring to FIG. 17A, the proximal structure (560) is an expandable compartment, comprising of reconfigurable element (410), supporting element (420) and control element (10). The proximal end of this structure can be joined in between the outer tube (460) and an inner tube (475). The distal tip of the control element (10) can be connected to the supportive element (450), and move freely in the inner tube (475) at the distal end of the proximal structure, as well as in pusher tube (20). By adjusting the supportive element through the control element, the radius and radial force of the distal structure can be adjusted. The distal structure (550) is in a form of basket or expandable compartment comprising a reconfigurable element (410) and enclosing element (425). In the middle of the distal structure, the tip of the enclosing element can be joined by connecter (451) to form a closed compartment/structure. The structure of the enclosing element can be the same as that of the supporting element described previously, but its function is just to close the compartment. Since the tip of the enclosing element is not connected to the control element, configuration and radial of the structure (550) cannot be adjusted. Alternatively, the distal structure can also be built without the enclosing element. The distal end can be closed by joining the distal wires/struts of the reconfigurable element (410) as illustrated in FIG. 17B (451). The proximal end of the distal structure (550) may be fixed to the distal tip of the control element. During retrieval process, the distal structure may catch clot debris that are unable to be contained by the proximal structure. The space between the two structures can also serve as a room to contain clot or clot debris during retrieval process.

A relatively the inner tubing (475) extending from the pusher tube tip/proximal connector into the middle of the proximal structure (560) can serve as adjustment tube 470 in FIG. 10 to prevent over expansion of distal expandable structure as illustrated in previous section.

In the embodiments which comprise two compartments, both the distal and proximal components can be made through laser cutting, photo etching, or wire braiding as disclosed in elsewhere of the application (e.g. FIG. 14A and related descriptions). Various materials described elsewhere can be used to make the reconfigurable element. In such embodiments, the strength/stiffness or wire/strut size of the proximal structure can be different from the distal structure. Further, the size of the distal structure (when it is fully expanded) may be different from that of the proximal structure.

A device which is configured to apply the embodiment comprising two expandable structures using design shown FIG. 16 as an example is illustrated in FIG. 18. In this particular embodiment, the distal compartment (550) and the proximal compartment (560) are employed in the device. Retrieval of a clot using the device according to FIG. 18 is illustrated in FIG. 19. The mechanism shown in FIG. 19 is merely an illustration of various applications, and presented as an illustration of certain embodiments. As discussed elsewhere in the application, the device according to the present application can be used to retrieve or remove clot in, for example, a blood vessel. Further, the device can be used to expand the luminal area and/or restore blood flow, which may or may not require retrieval of a clot.

In this hypothetical condition where a clot (60) is located in a blood vessel (50) as seen in FIG. 19 A, the microcatheter (30) comprising the retrieval device can be navigated and located in close proximity to the clot or distal to the clot. The microcatheter (30) then is unsheathed to expose clot to the retrieval device. In some embodiments shown in FIG. 19, a clot can be engaged and retrieved through the following mechanisms:

a clot (60) can be held between the proximal structure (560) and the tip of micro-catheter (30) and removed from the original position (FIG. 19B).

a clot (60) can be held in between the proximal structure (560) and the distal structure (550) and removed from the original position (FIG. 19C).

a clot (60) can be held/engaged by the proximal structure (560) and removed from the original position (FIG. 19D). In some cases, the clot can be engaged between the proximal structure and the artery wall and removed with friction between the clot and the device.

a clot (60) can be held/engaged by the distal structure (550) and removed from the original position (FIG. 19E). In some cases, the clot can be engaged between the distal structure and the artery wall and removed with friction.

a clot (60) can be broken-up into debris from the proximal structure. They may fall into, or be caught by the distal structure (550) and/or the room between proximal structure (560) and distal structure (550) (FIG. 19F).

a clot (60) can be engaged at various location/points and removed through combinations of any above mechanisms (FIG. 19G).

According to some embodiments, during the clot engagement and, or retrieval process, the control element (10) can be pulled back at any time to maneuver the radial force and radius (i.e. size) of the retrieval device to ensure that the clot is engaged with the device and not slide away from the device (FIG. 19H).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A device for use in a body lumen comprising:
a pusher tube and an expandable compartment;
wherein the expandable compartment comprises:
a control element comprising a proximal end and a distal end;
a reconfigurable element, said reconfigurable element being associated with a supportive element and the control element; and
a supportive element, said supportive element being associated with the control element and the reconfigurable element, wherein the supportive element is configured to adjust a radial force and a configuration of the reconfigurable element.

2. The device according to claim 1, wherein the control element comprises a wire, cable, or braid.

3. The device according to claim 1, wherein the reconfigurable element and/or the supportive element comprise(s) a plurality of wires.

4. The device according to claim 3, wherein the reconfigurable element can self expand into a relaxed expandable state to form a compartment or basket.

5. The device according to claim 4, wherein the reconfigurable element comprises a plurality of cells, and a size of a cell and a thickness of wires surrounding the cell vary within the reconfigurable element.

6. The device according to claim 1, wherein the supportive element is in a form of strut that is manufactured from a same piece of material as the reconfigurable element and automatically connected to the reconfigurable element.

7. The device according to claim 1, wherein the supportive element is in a form of wire mesh or a braid.

8. The device according to claim 1, wherein the pusher tube is connected to the expandable compartment.

9. The device according to claim 1, wherein the control element is surrounded by the pusher tube and can move freely inside the pusher tube.

10. The device according to claim 1, wherein the supportive element comprises a first configuration and a second configuration, said first configuration having a smaller angle between the supporting element and the control element than the angle in the second configuration.

11. The device according to claim 1, wherein the supportive element comprises a first configuration and a second configuration, said first configuration having an outer diameter which is smaller than an outer diameter of the second configuration.

12. The device according to claim 10 and 11, wherein projection of the control element to a distal direction causes transition of the supportive element to the first configuration such that the radial force of the reconfigurable element is reduced.

13. The device according to claim 10 and 11, wherein projection of the control element to a proximal direction causes transition of the supportive element to the second configuration such that the radial force of the reconfigurable element is increased.

14. The device according to claim 1, wherein the device is configured to remove an occlusion blocking a blood vessel, to open a blocked section of a blood vessel and/or to increase a flow in a blood vessel.

15. The device according to claim 1, wherein a distal end of the reconfigurable element is joined forming a closed-end reconfigurable element, or not jointed forming an open-end reconfigurable element.

16. The device according to claim 1, wherein the sides of the reconfigurable element are joined forming a closed-sided reconfigurable element, or not joined forming an open-sided reconfigurable element.

17. The device according to claim 1, wherein the supportive element comprises a plurality of wires, and said wires are extendible between the proximal and distal ends, and substantially surrounded by the reconfigurable element.

18. The device according to claim 1, wherein the reconfigurable element and the supportive element are in a form of wire mesh, which can be extendible between the proximal and distal ends, and the supportive element is substantially surrounded by the reconfigurable element, thereby forming a double-layered reconfigurable element.

19. The device according to claim 1, wherein the reconfigurable element comprises a plurality of linear wires aligned substantially in parallel and a plurality of wires in a substantially circular form, and the supportive element comprises at least two wires that is associated with the reconfigurable element and the control element, thereby forming an umbrella-shaped expandable compartment.

20. The device according to claim 1, wherein a distal end of the reconfigurable element and a distal end of the supportive element are not connected and move independently.

21. The device according to claim 1, wherein an atraumatic flexible coil is attached to the distal tip of the reconfigurable element.

22. The device according to claim 1, wherein the device comprises a distal expandable structure and a proximal expandable structure.

23. The device according to claim 22, wherein the first expandable structure comprises a reconfigurable element, a control element, and optionally a supportive element.

24. The device according to claim 22, wherein said the second expandable structure comprises a reconfigurable element and optionally an enclosing element.

25. The device according to claim 22, wherein a distance between the distal and proximal expandable structures is adjustable.

26. A method of removing an occlusion present in a first position of a blood vessel comprising:
introducing the device according to claim 1 into the blood vessel;
locating the device at the first position of the blood vessel;
adjusting the radial force and/or configuration of the reconfigurable element of the device; and
removing the occlusion from the first location.

27. The method according to claim 26 wherein removing the occlusion further comprises one or more selected from the group consisting of:
engaging the occlusion at least partially with the device;
disassembling the occlusion into small-sized debris and collecting at least part of the debris; and
expanding the area of the blood vessel.

28. The method according to claim 26 wherein the method is configured to be applied for treatment of stroke.

29. A method of increasing a flow in a blood vessel comprising:
introducing the device according to claim 1 into the blood vessel;
locating the device at about the first position of the blood vessel that is in need of increasing the flow; and
adjusting the radial force and/or configuration of the reconfigurable element of the device so as to expand an area of the first position.

30. A method of removing an occlusion present in a first position of a blood vessel comprising:
introducing a device according to claim 1 into the blood vessel;
locating the device at about the first position of the blood vessel;
supporting the expanded status of the reconfigurable element by extending a support element associated with the reconfigurable element by proximal movement of the control element;
further supporting a more expanded status of the reconfigurable element by extending a support element associated with the reconfigurable element by proximal movement of the control element;
grabbing the occlusion with the reconfigurable element in its expanded status;
shifting the configuration of the reconfigurable element toward a more relaxed status by distally moving the control element; and
removing the occlusion from the first location.

31. A method of removing an occlusion present in a first position of a blood vessel comprising:
introducing a device according to claim 22 into the blood vessel;
locating the device distal to the position of the blood vessel;
pulling the pusher tube proximally to grab the occlusion between the two expandable structures in its expanded status;
supporting the expanded status of the reconfigurable element by extending a support element associated with the reconfigurable element by proximal movement of the control element;
grabbing the clot with the proximal expandable structure;
catching the clot debris with the distal expendable structure;
optionally retrieving the device while pulling the control element to increase the radial force of the expendable structure and further support an expanded status of the expandable structure; and
removing the occlusion from the first location.
